(19) United States
(12) Patent Application Publication
(10) Pub. No.: US 2014/0378965 A1
(43) Pub. Date: Dec. 25, 2014

(54) ELECTROSURGICAL ELECTRODE

(71) Applicant: GYRUS MEDICAL LIMITED, Cardiff (GB)

(72) Inventor: Anthony K. ATWELL, Newport (GB)

(21) Appl. No.: 14/308,367

(22) Filed: Jun. 18, 2014

(30) Foreign Application Priority Data
Jun. 24, 2013 (GB) 1311194.3

(51) Int. Cl. A61B 18/14

(52) U.S. Cl. CPC A61B 18/149; A61B 2018/00505
USPC 606/41

(57) ABSTRACT

An electrode for vaporising tissue comprises a head (26) supported by at least one externally insulated electrically conducting stem (27). The head (26) is typically a button electrode and is provided with a conductive portion (41) in which the surface of the button electrode is electrically conductive, and an insulated portion (37) in which the surface of the button electrode is non-conductive. The head is typically mounted on the stem (27) such that it is capable of rotating about the stem such that it can change its rotational orientation depending on the direction of movement of the electrode. Typically, the head (26) is asymmetrical such that it has a greater width in one direction as compared with another direction, such as having an oval cross-section. The head (26) is rotatable about the stem (27) such that, whichever the direction of movement of the electrode, the electrode orients itself such that it presents the greater width parallel to the direction of movement of the electrode.

### TECHNICAL FIELD

This invention relates to an electrosurgical electrode and in particular to an electrode and working element for use in a system for endoscopic urological surgery using a resectoscope.

### BACKGROUND TO THE INVENTION AND PRIOR ART

Electrosurgical systems for endoscopic urological surgery are well known in the art, examples being given in U.S. Pat. Nos. 5,007,907 and 6,322,494. Such systems include an electrosurgical instrument deployable by means of a resectoscope, and an electrosurgical generator powering the instrument. A generator suitable for powering a urological instrument is described in U.S. Pat. No. 7,211,081. Instruments used in electrosurgical urology surgery are either bipolar, in which case two electrodes are present at the distal end of the instrument, or monopolar, in which case one electrode is present on the instrument and a second electrode is provided in the form of a patient return plate.

Different types of electrodes have been used previously depending on their intended function. Loop electrodes (see U.S. Pat. No. 4,917,082 as an example) are generally good for tissue resection, while roller electrodes (see U.S. Pat. No. 5,549,605 as an example) or slider electrodes (see U.S. Pat. No. 5,766,168 as an example) are used for vaporisation and/or coagulation of tissue.

### SUMMARY OF THE INVENTION

Embodiments of the present invention provide a different electrode capable of efficient tissue resection and vaporisation/coagulation. In particular, embodiments of the present invention provide a button electrode having a tissue treatment surface that is divided into at least a first portion and a second portion, the first portion being electrically conductive so as to provide a conductive portion and the second portion being electrically insulating so as to provide an insulated portion. In some embodiments the button electrode is divided along a chord line into the first and second portions, with the first portion forming one segment of the button electrode, and the second portion forming another segment of the button electrode. The button electrode is rotatably mounted on a stem, that provides connection in use to a source of radio frequency (RF) electrosurgical signals, and is further arranged such that when in use and in contact with tissue movement of the button electrode in a direction of movement causes the button electrode to rotate so as to present the conductive portion to face the direction of movement with the insulating portion trailing behind the conductive portion. With such an arrangement a conductive button electrode can be provided capable of the controllable coagulation or vaporisation of tissue as required, but with a reduced conductive surface area, because of the insulating portion, which in turn reduces the current through the electrode and hence reduces unwanted secondary heating effects due to the current.

Accordingly, from one aspect there is provided an electrode for vaporising tissue comprising an elongate conductive lead, the lead defining an axis having a proximal direction and a distal direction, a stem depending from the lead at an angle to the axis, and a head supported by the stem, the head being in the form of a button electrode and being provided with a conductive portion in which the surface of the button electrode is electrically conductive, and an insulated portion in which the surface of the button electrode is non-conductive, the electrode being such that when it is moved in the distal direction in contact with tissue the insulated portion is wholly proximal of the conductive portion.

Therefore, when the electrode is moved in the distal direction when in contact with tissue, the conductive portion faces the distal direction and the insulated portion trails the conductive portion, facing the proximal direction.

The non-conductive portion is provided either by coating a portion of the head with an electrically insulating material, or by forming part of the button electrode from an electrically insulating material. In this way, the electrically conductive portion of the button electrode is still capable of the vaporisation or coagulation of tissue as desired, while the insulated portion reduces the current emanating from the electrode. By reducing the current in this way, overheating of the electrode, or, in the case of a wet field operation, any saline surrounding the electrode, is avoided.

In one convenient arrangement, the button electrode is hemispherical in shape. Alternatively, the button electrode is asymmetrical such that it has a greater width in one direction as compared with another direction. Conceivably, the button electrode has an oval cross-section in plan view. The stem is conveniently formed of an electrically conducting material, such that it can act as a lead to transmit RF energy to the button electrode. The stem is conceivably provided with a covering of electrically insulating material.

Typically, the button electrode is mounted on the stem such that it is capable of rotating about the stem such that it can change its rotational orientation depending on the direction of movement of the electrode. Typically, the electrode is such that when it is moved in the proximal direction in contact with tissue the electrode rotates such that the insulated portion is wholly distal of the conductive portion. Therefore, when the electrode is moved in the proximal direction when in contact with tissue, the conductive portion faces the proximal direction and the insulated portion trails the conductive portion, facing the distal direction. In this way, the button electrode can always present whichever orientation is most effective for the treatment of tissue, regardless of the direction of movement of the electrode. Conveniently, the button electrode is rotatable about the stem such that, whichever the direction of movement of the electrode, the electrode orients itself in use such that it presents the greater width transverse to the direction of movement of the electrode. In this way, whichever way the electrode is moved, the button electrode will orient itself in use such that the greater width (the major axis in the case of an oval electrode) lies transverse to the axis of movement of the electrode. This provides a larger area of tissue treatment as the electrode is moved across or through tissue.

The swivelling of the button electrode in use is typically due to the drag of the electrode against tissue, causing the electrode to re-orient itself. Conceivably, the movement of the button electrode through a conductive fluid such as saline may be sufficient to cause enough drag as to be able to re-orient the electrode, such that it becomes aligned even before coming into contact with tissue. It may be advantageous to provide the button electrode with some form of resistive member such as a vane or paddle to assist with such reorientation.

Conveniently, the button electrode is mounted eccentrically on the stem such that the stem meets the head off centre. Typically, the button electrode has a major and a minor axis, and the stem meets the button electrode on the minor axis at a point between one side of the electrode and the centre point where the major axis meets the minor axis. In this way, the button electrode will swivel in use such that the major axis is presented transverse to the direction of movement, so as to create the largest possible profile for the treatment of tissue.

Conveniently, the button electrode is rotatable about the stem such that, whichever the direction of movement of the electrode, the electrode orients itself in use such that the insulated portion is longitudinally aligned with respect to the direction of movement of the electrode. In this way, the electrode tends to align itself such that the insulated portion is located behind the electrically conductive remainder of the button electrode. As the electrode is moved, the part of the electrode coming into contact with tissue is the electrically conducting portion of the button electrode, followed afterwards by the insulated portion. Thus, the electrically conductive portion of the button electrode is still capable of the vaporisation or coagulation of tissue as desired, while the insulated portion reduces the current emanating from the electrode. By reducing the current in this way, overheating of the electrode, or, in the case of a wet field operation, any saline surrounding the electrode, is avoided.

According to another convenient arrangement, the button electrode is provided with an additional coagulation electrode separated from the remainder of the button electrode by the insulated portion. The generator supplying current to the electrode is switched such that when the electrode is being used to vaporise tissue, the conductive portion of the button electrode is connected to the generator. Conversely, when the electrode is being used to coagulate tissue, the additional coagulation electrode of the button electrode is connected to the generator. In either arrangement, current passes from the button electrode to a return electrode, either located nearby as a bipolar return electrode or as a remote patient plate in a monopolar arrangement. Preferably, the electrode orients itself in use such that the coagulation electrode is at the rear of the button electrode as it is disposed with respect to the direction of movement of the electrode. This is the case whichever way the electrode is moved, with the button electrode re-orienting itself automatically due to the drag of the head against the tissue.

According to a further aspect of the invention there is provided a working element for a resectoscopic electrosurgical instrument, the working element comprising a handle, an elongate shaft extending from the handle and defining an axis having a proximal direction and a distal direction, and an electrode received within the shaft and comprising an elongate conductive lead, the lead having a stem depending from the lead at its distal end at an angle to the axis, and a head supported by the stem, the head being in the form of a button and being provided with a conductive portion in which the surface of the button electrode is electrically conductive, and an insulated portion in which the surface of the button electrode is non-conductive, the electrode being such that the insulated portion is wholly proximal of the conductive portion.

From another aspect embodiments of the invention further provide a button electrode for an electrosurgical instrument, the button electrode having a tissue treatment surface that is divided into at least a first portion and a second portion, the first portion being electrically conductive so as to provide a conductive portion and the second portion being electrically insulating so as to provide an insulated portion, the button electrode being rotatably mounted on a stem that provides connection in use to a source of radio frequency (RF) electrosurgical signals, the button electrode being further arranged such that when in use and in contact with tissue movement of the button electrode in any direction of movement causes the button electrode to rotate so as to present the conductive portion to face the direction of movement with the insulating portion wholly trailing behind the conductive portion.

In one embodiment the button electrode is divided along at least one chord line into the first and second portions, with the first portion forming one segment of the button electrode, and the second portion forming another segment of the button electrode.

In one embodiment the electrode is substantially elliptical in shape, the insulating portion being located all to one side of the major axis of the substantially elliptical electrode. The elliptical shape in particular can help the electrode to self-orient itself so as to present the conductive portion to the direction of movement. In order to aid in this self-orienting operation, the button electrode may be eccentrically rotatably mounted on the stem.

In a further embodiment the tissue treatment surface is further divided into a third portion, the third portion being electrically conductive and separated from the first portion by the second portion, the arrangement being such that in use the third portion wholly trails behind the second portion in the direction of movement. In some embodiments the third portion may be used to provide a coagulating RF waveform, or alternatively may be used as the return electrode, or part of the return electrode, in a bipolar arrangement.

### BRIEF DESCRIPTION OF THE DRAWINGS

Embodiments of the invention will now be further described, by way of example only, with reference to the accompanying drawings, in which:

FIG. 1 is a perspective view of an electrosurgical system for use with an electrode in accordance with an embodiment of the present invention,

FIG. 2 is an exploded view of a resectoscopic instrument used as part of the electrosurgical system of FIG. 1,

FIG. 3 is a side view of an electrode in accordance with an embodiment of the present invention,

FIG. 4 is an enlarged view of a part of the electrode of FIG. 3,

FIG. 5 is a plan view of the electrode of FIG. 3,

FIG. 6 is an enlarged view of the electrode of FIG. 4 being moved in the opposite direction, and

FIG. 7 is a side view of an alternative embodiment of electrode in accordance with an embodiment of the present invention.

### DESCRIPTION OF THE EMBODIMENTS

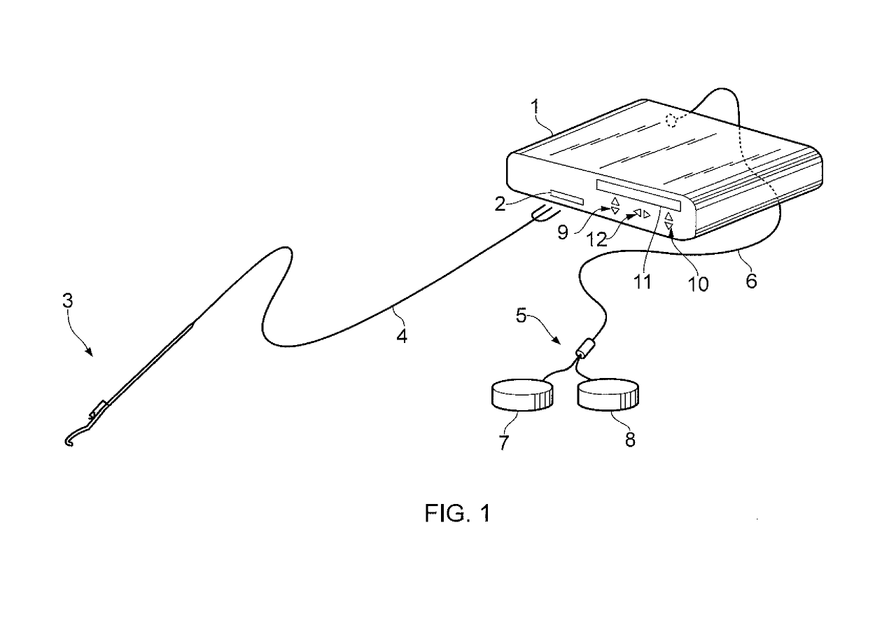

Referring to FIG. 1, a generator 1 has an output socket 2 providing a radio frequency (RF) output for an instrument 3 via a connection cord 4. Activation of the generator may be performed from the instrument 3 via a connection in cord 4 or by means of a footswitch unit 5, as shown, connected to the rear of the generator by a footswitch connection cord 6. In the illustrated embodiment footswitch unit 5 has two footswitch pedals 7 and 8 for selecting a coagulation mode and a cutting/vaporisation mode of the generator respectively. The generator front panel has push buttons 9 and 10 for respectively setting coagulation and cutting/vaporisation power levels, which are indicated in a display 11. Push buttons 12 are provided as a means for selection between alternative coagulation and cutting/vaporisation waveforms.

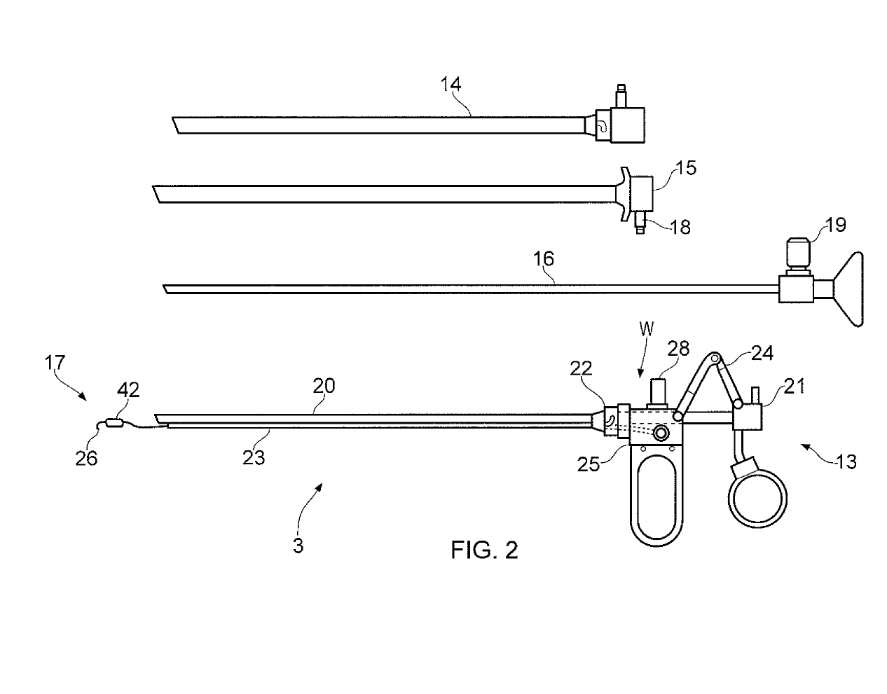

As shown in FIG. 2, the instrument 3 is deployed through a resectoscope 13 including an inner sheath 14, an outer sheath 15, and a rod lens telescope/light source assembly 16. The instrument 3 is part of a working element, indicated generally by the reference W and including an electrode assembly 17.

The sheaths 14 and 15 provide for the supply and aspiration of an operating site with a fluid medium via a connector 18. The outer sheath 15 locks over the inner sheath 14, forming a watertight seal. Typically, the inner sheath 14 has a diameter of 24Fr (8 mm), and the outer sheath 15 has a diameter of 27 Fr (9 mm). The telescope assembly 16 provides the means of illuminating and viewing the operative site via a light source (not shown) connected thereto by a connector 19. The viewing angle of the telescope is generally at 30° to its axis.

The working element W may be either passive or active, that is to say the cutting stroke of the electrode may be as the result of a spring bias or against the force of a spring bias. The telescope assembly 16 includes a telescope support tube 20 having a telescope connector 21 at its proximal end, and a sealing block 22 located part way along the support tube 20, the inner sheath 14 being connected to the sealing block. Both of these interfaces are watertight. An electrode support tube 23 is attached to the underside of the telescope support tube 20 on the distal side of the sealing block 22 for the majority of its length. Two spring-loaded links 24 and an insulation block 25, located between the sealing block 22 and the telescope connector 21, make up the mechanism. The active mechanism is arranged so that the spring-loaded links 24 assist the forward stroke, while, in the passive version the links aid the backward stroke. In general, the range of travel is about 25 mm.

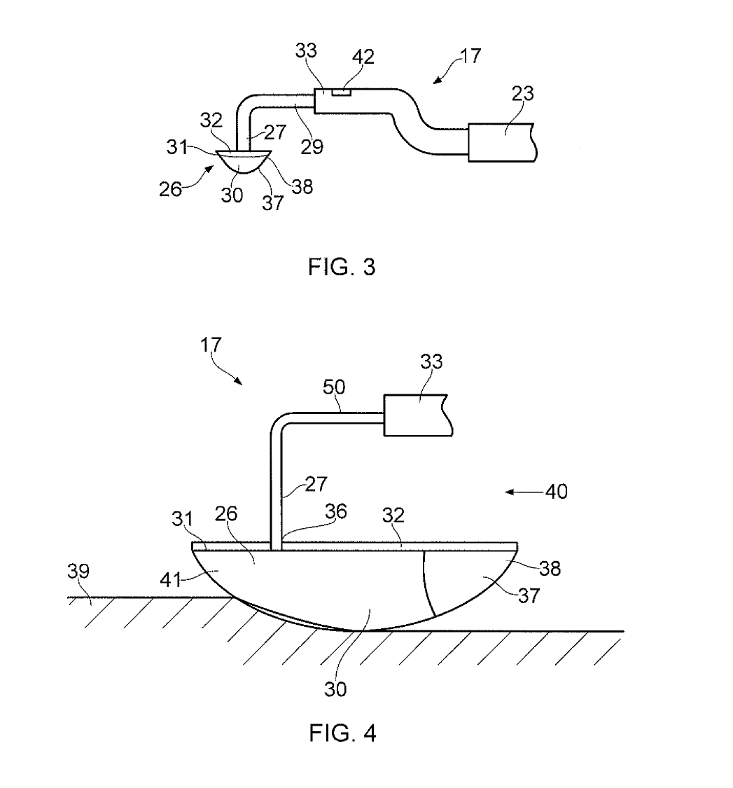

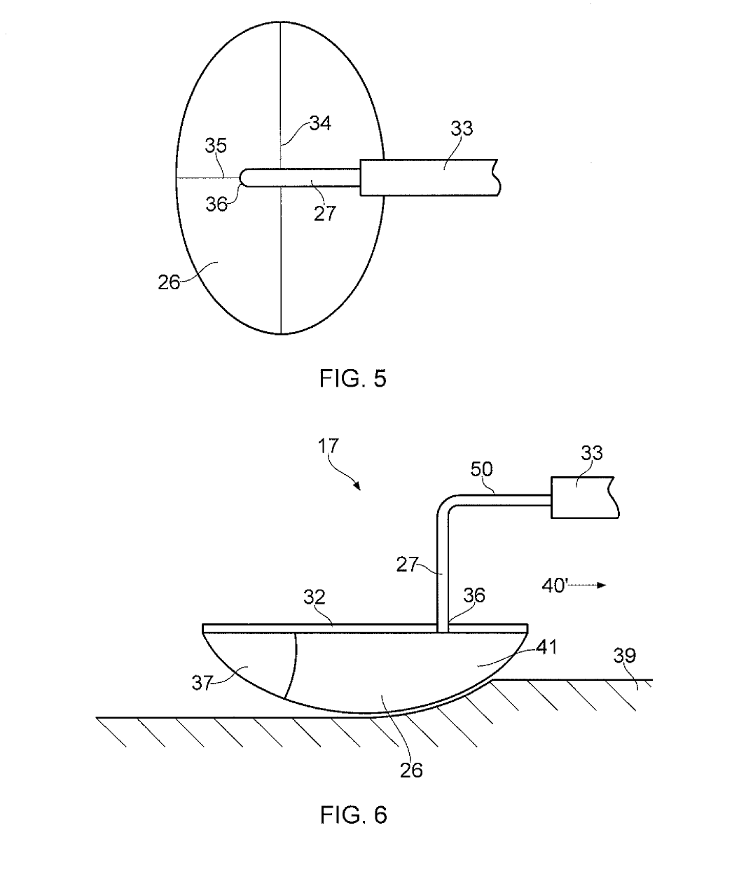

Relevant parts pertinent to embodiments of the present invention, and in particular the electrode assembly 17 will now be described in more detail. The electrode assembly 17 is connected to the generator 1 via cord 4 connected via socket 28. Referring to FIG. 3, the electrode assembly 17 comprises an electrode head 26 made of an appropriate high-temperature resistant metal. The electrode head 26 is mounted on a stem 27 connected to an elongate feed conductor 50 covered with an insulating, plastic sheath 29. The feed conductor 50 emerges from a protective tube 33, which feeds into the electrode support tube 23 of FIG. 2. The electrode head 26 assumes the shape of a mushroom with a strongly convex functional surface 30. Its upper surface 31 is planar and covered by a ceramic layer 32 which is crossed by the feed conductor inside the stem 27 passing through a borehole (not shown). The head 26 has a planar upper surface 31 as before, and an insulating ceramic layer 32 covering the upper surface 31. The head 26 also has an insulated portion 37 in which the metallic head is covered with an insulating covering 38.

FIGS. 4 & 5 illustrate an alternative embodiment of electrode assembly 17, in which the head 26 has an oval configuration when viewed from above. The oval head 26 has a major axis 34 and a minor axis 35. The electrode head 26 is mounted on the stem 27 such that it can rotate about the stem and assume any angular orientation. The head 26 is rotatably mounted on the stem 27 at a point 36 located on the minor axis to one side of the midpoint intersection with the major axis 34.

The rotatable mounting of the head 26 on the stem 27 is such that when the electrode assembly 17 is moved against tissue 39 in the direction of the arrow 40, the head 26 rotates about the stem 27 such that the major axis 34 lies transverse to the direction of movement. Moreover, the rotation of the head 26 is such that the insulated portion 37 lies proximally of the stem 27, while the un-insulated remainder of the head forms an active electrode 41 distal of the insulated portion 37. This active electrode 41 is capable of the vaporisation or coagulation of the tissue 39, while the insulated portion 37 (which plays no part in the vaporisation or coagulation of the tissue) reduces the amount of current emitted by the head 26. This reduction in current output helps to avoid the overheating of the head 26, or any conductive fluid (not shown) surrounding the electrode assembly 17.

Alternatively, and as shown in FIG. 6, when the electrode assembly 17 is moved against tissue 39 in the opposite direction, i.e. in the direction of the arrow 40′, the head 26 rotates about the stem 27 such that the insulated portion 37 lies distally of the stem 27, with the un-insulated remainder of the head forming an active electrode 41 proximal of the insulated portion 37. Thus, regardless of whether the electrode assembly is moved in a distal direction or a proximal direction, the head always rotates such that the insulated portion 37 is to the rear of the active electrode 41 with respect to the direction of movement.

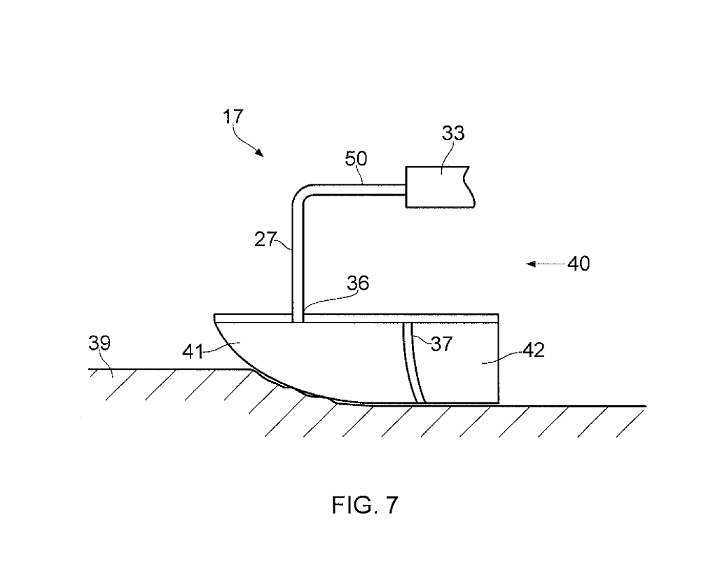

The active electrode 41 can be used as a monopolar electrode in conjunction with a remote patient return plate (not shown), or alternatively in a bipolar configuration in conjunction with a return electrode (not shown) present on the stem 27 or tube 33. An alternative configuration is shown in FIG. 7, in which an active electrode 41, an insulated portion 37 and an additional coagulation electrode 42 are all present on the head 26. When the vaporisation of tissue is required, the generator 1 supplies RF energy to the active electrode 41. Conversely, when the coagulation of tissue is required, the generator 1 supplies RF energy to the coagulation electrode 42. In this way, a separate electrode optimised for either vaporisation or coagulation is used in each case.

The head 26 is rotatably mounted on the stem 27 as previously described, with the mounting point 36 being offset from the midpoint intersection with the major axis, also as previously described. In this way, when the electrode assembly 17 is moved against tissue 39 in the direction of the arrow 40, the head 26 rotates about the stem 27 such that the active electrode 41 lies distal of the coagulation electrode 42. Conversely, when the electrode assembly is moved against tissue 39 in the opposite direction, the head 26 rotates about the stem 27 such that the active electrode 41 lies proximal of the coagulation electrode 42. The active electrode 41 is therefore always presented to the tissue, to perform tissue vaporisation when required.

Alternative embodiments to those described above will be apparent to those skilled in the art without departing from the scope of the present invention. Different shapes of head and electrode configuration can be used, with the common inventive features being that the electrode head is partially insulated and is rotatably mounted such that it can re-orient itself to present to the tissue the most effective constructions or profile.

1. An electrode for vaporising tissue comprising an elongate conductive lead, the lead defining an axis having a proximal direction and a distal direction, a stem depending from the lead at an angle to the axis, and a head supported by the stem, the head being in the form of a button and being provided with a conductive portion in which the surface of the button electrode is electrically conductive, and an insulated portion in which the surface of the button electrode is non-conductive, the electrode being such that when it is moved in the distal direction in contact with tissue the insulated portion is wholly proximal of the conductive portion.

2. An electrode according to claim 1, wherein the button electrode is hemispherical in shape.

3. An electrode according to claim 1, wherein the button electrode is asymmetrical such that it has a greater width in one direction as compared with another direction.

4. An electrode according to claim 3, wherein the button electrode has an oval cross-section in plan view.

5. An electrode according to claim 1, wherein the button electrode is mounted on the stem such that it is capable of rotating about the stem such that it can change its rotational orientation depending on the direction of movement of the electrode.

6. An electrode according to claim 5, wherein the electrode is such that when it is moved in the proximal direction in contact with tissue the electrode rotates such that the insulated portion is wholly distal of the conductive portion.

7. An electrode according to claim 5, wherein the button electrode is asymmetrical such that it has a greater width in one direction as compared with another direction, and the button electrode is rotatable about the stem such that, whichever the direction of movement of the electrode, the electrode orients itself such that it presents the greater width transverse to the direction of movement of the electrode.

8. An electrode according to claim 7, wherein the button electrode is mounted eccentrically on the stem such that the stem meets the button electrode off centre.

9. An electrode according to claim 8, wherein the button electrode has a major and a minor axis, and the stem meets the button electrode on the minor axis at a point between one side of the electrode and the centre point where the major axis meets the minor axis.

10. An electrode according to claim 1, wherein the button electrode is rotatable about the stem such that, whichever the direction of movement of the electrode, the electrode orients itself such that the insulated portion is longitudinally aligned with respect to the direction of movement of the electrode.

11. An electrode according to claim 1, wherein the button electrode is provided with an additional coagulation electrode separated from the remainder of the button electrode by the insulated portion.

12. An electrode according to claim 11, wherein the electrode orients itself such that the coagulation electrode is at the rear of the button electrode as it is disposed with respect to the direction of movement of the electrode.

13. An electrode according to claim 1, wherein the stem is formed from an electrically conducting material.

14. An electrode according to claim 13, wherein the stem is provided with a covering of electrically insulating material.

15. A working element for a resectoscopic electrosurgical instrument, the working element comprising a handle, an elongate shaft extending from the handle and defining an axis having a proximal direction and a distal direction, and an electrode received within the shaft and comprising an elongate conductive lead, the lead having a stem depending from the lead at its distal end at an angle to the axis, and a head supported by the stem, the head being in the form of a button and being provided with a conductive portion in which the surface of the button electrode is electrically conductive, and an insulated portion in which the surface of the button electrode is non-conductive, the electrode being such that the insulated portion is wholly proximal of the conductive portion.

16. A button electrode for an electrosurgical instrument, the button electrode having a tissue treatment surface that is divided into at least a first portion and a second portion, the first portion being electrically conductive so as to provide a conductive portion and the second portion being electrically insulating so as to provide an insulated portion, the button electrode being rotatably mounted on a stem that provides connection in use to a source of radio frequency (RF) electrosurgical signals, the button electrode being further arranged such that when in use and in contact with tissue movement of the button electrode in any direction of movement causes the button electrode to rotate so as to present the conductive portion to face the direction of movement with the insulating portion trailing behind the conductive portion.

17. A button electrode according to claim 16, wherein the button electrode is divided along a chord line into the first and second portions, with the first portion forming one segment of the button electrode, and the second portion forming another segment of the button electrode.

18. A button electrode according to claim 16, wherein the electrode is substantially elliptical in shape, the insulating portion being located to one side of the major axis of the substantially elliptical electrode.

19. A button electrode according to claim 16, wherein the button electrode is eccentrically rotatably mounted on the stem.

20. A button electrode according to claim 16, wherein the tissue treatment surface is further divided into a third portion, the third portion being electrically conductive and separated from the first portion by the second portion, the arrangement being such that in use the third portion trails behind the second portion in the direction of movement.
